(19) United States
(12) Patent Application Publication
(10) Pub. No.: US 2025/0235143 A1
(43) Pub. Date: Jul. 24, 2025

(54) A PORTABLE ECG DEVICE WITH ONLINE AND OFFLINE MODES AND A PROCESS FOR ECG DATA ANALYSIS

(71) Applicant: QUORETECH LLC, Wilmington, DE (US)

(72) Inventors: Carlos Tadeu BASSI, Santa Paula São Caetano do Sul (BR); Aleksandr VNUKOV, Kharkiv (UA); Guilherme de Freitas NEMETH, Mauá (BR); Marcelo Nachbar da SILVA, São Paulo (BR); Marcio Yudi KAKUMOTO, São Paulo (BR); Marcos Roberto Cobbra e SILVA, Mauá (BR); Marcos Vinicius PINTO, São José do Rio Preto (BR); Ricardo Kouiti Santos IGUCHI, Ribeirão Preto (BR); Samuel Pereira Mesquita DE SOUSA, Sao Paulo (BR); Tomasz ROSZCZYNIALSKI, Krakow (PL)

(21) Appl. No.: 18/703,511

(22) PCT Filed: Oct. 20, 2021
(86) PCT No.: PCT/US2021/055850

§ 371 Date: Apr. 22, 2024

(51) Int. Cl. A61B 5/332; A61B 5/00; A61B 5/333; A61B 5/339

(52) U.S. Cl. CPC A61B 5/332; A61B 5/0006; A61B 5/333; A61B 5/339; A61B 5/7267; A61B 2560/0219; A61B 2560/0456; A61B 2560/0475; A61B 2562/182

(57) ABSTRACT

This application relates in general to a portable ECG monitoring and, in particular, to a process for ECG signal analysis using an artificial intelligence backend software with machine-learning-based with a reinforcement learning algorithm. This invention presents a waterproof portable device for gathering and monitoring of ECG data of an individual, configured to work on either online or offline mode. The process comprises at least the steps of (a) preparing an ECG exam through a first device containing a software; (b) connecting the first device to a portable ECG device; (c) setting up the portable ECG device on a patient's chest; (d) collecting the ECG data; (e) uploading the data to an artificial intelligence backend software; (f) processing and classifying the data using a machine learning algorithm; (g) analyzing the classification provided in step “f” and performing a second classification of data; and (h) providing a signed medical report.

### INVENTION FIELD

This application relates in general to a portable ECG monitoring and, in particular, to a process for ECG signal analysis using an artificial intelligence backend software with machine-learning-based/deep-learning with a reinforcement learning algorithm.

### BACKGROUND

A typical electrocardiogram (ECG) records electrical potentials in the heart using a standardized set format 12-lead configuration to collect and record cardiac electrical signals. Among the variations of an ECG, one can find a single Lead, 3-Lead, 5-Lead, 18-Lead or even a 22-Lead, mostly based on a post-processing of a 12-Lead ECG algorithm. The analysis of a single Lead or a single resultant ECG signal neglects nuances that can only be found when considering the Leads themselves.

The document BR102020007000 (henceforth BR10) presents an arrhythmia diagnostic system and method useful to aid in the diagnosis of cardiac arrhythmias, being applied to cardiac monitoring equipment. More specifically, BR10 refers to a remote, mobile and non-invasive cardiac monitoring system, which uses hardware, software and backend support to perform autonomous and remote monitoring, using an artificial intelligence system to analyze and compose pre-diagnostics, and said invention provides more speed, security and accuracy to diagnoses obtained from data collected through cardiac monitoring equipment, as well as refers to the method of diagnosing cardiac arrhythmias. The analysis method of BR10 consists of three diagnoses, each diagnosis being generated by an algorithm that has undergone specific training. More specifically, an algorithm is trained by the professional responsible for that patient's ECG, an algorithm is trained by the contribution of the reports provided by each of the professionals registered in the system, and a third algorithm is trained based on the reports provided by a subgroup of registered professionals in the system, with this subgroup formed by professionals considered reference in the production of diagnoses of cardiac arrhythmias. It is not obvious from BR10 that ECG analysis can be performed on each Lead individually. More than that, the comparison of results in the current invention is performed within the analysis of the Leads of that ECG, offering a more accurate identification of anomalies. For example, an anomaly can be seen in Lead II that was not evident in Leads I and III and is not evident from the ECG result. Besides, BR10 does not contain the intricate and optimized process present in the current invention.

A system for electrocardiogram (ECG) interpretation of U.S. Pat. No. 9,895,075 (henceforth US9) includes a processor and memory coupled to the processor. A rendering module is stored in the memory and is configured to receive input from an ECG and correlate interpretation statements with ECG measurements responsible for the interpretation statements in the ECG. An interface is configured to permit user selection of at least one interpretation statement in a display, the display being configured to render visual indications of the ECG measurements on digitally rendered waveforms of the ECG. The electrocardiographic data that are processed according to the US9 system generate digitally rendered signal waves, these waves being scalable, so that the visualization is optimized for each device used. For example, the images of signal waves can be viewed on the screen of a smartphone, on a computer monitor, on a tablet and any other device with a digital image display screen, with no image distortion or distortion regardless of the device used. Also, these images allow the inclusion of texts, images or even audio in selected portions of the wave, and these insertions are notes of interest to the physician responsible for the report. However, the system of US9 does not undergo training nor contains a reinforcement learning algorithm. Also, different from US9, the current invention gives the medical doctor a choice on the algorithm to use on the analysis.

The document U.S. Pat. No. 10,463,269 (henceforth US10) provides a system and method for machine-learning based atrial fibrillation detection. A database is maintained that is operable to maintain a plurality of ECG features and annotated patterns of the features. At least one server is configured to: train a classifier based on the annotated patterns in the database; receive a representation of an ECG signal recorded by an ambulatory monitor recorder during a plurality of temporal windows; detect a plurality of the ECG features in at least some of the portions of the representation falling within each of the temporal windows; use the trained classifier to identify patterns of the ECG features within one or more of the portions of the ECG signal; for each of the portions, calculate a score indicative of whether the portion of the representation within that ECG signal is associated the patient experiencing atrial fibrillation; and take an action based on the score. Even though US10 covers part of the deficiencies usually found in the state of the art, it lacks variety by considering a single Lead ECG analysis. Besides, the algorithm chosen by US10 does not learn through reinforcement method, but through a pre-set analysis with ECG data already existent in ambulatory ECG monitors. This setting does not allow an individualized analysis, i.e., an algorithm that is personalized for a specific group. For example, US10 uses the same algorithm to evaluate the ECG signal of both a 40 years old smoker and a 19 years old athlete.

A system and method for medical premonitory event estimation according to EP 3 204 881 (henceforth EP3) includes one or more processors to perform operations comprising: acquiring a first set of physiological information of a subject, and a second set of physiological information of the subject received during a second period of time; calculating first and second risk scores associated with estimating a risk of a potential cardiac arrhythmia event for the subject based on applying the first and second sets of physiological information to one or more machine learning classifier models, providing at least the first and second risk scores associated with the potential cardiac arrhythmia event as a time changing series of risk scores, and classifying the first and second risk scores associated with estimating the risk of the potential cardiac arrhythmia event for the subject based on the one or more thresholds. EP3 does not include the patient or ECG wearer as an active element in the ECG collecting process, leaving the medical doctor to the habitual hand-comparing of an ECG signal and the patient's notes of symptoms or activities. Also, EP3 does not present itself as an everyday system, nor as applicable to a portable ECG device with wireless connectivity. At last, the algorithm is not customizable to each individual or group of individuals that share the same cardiac relevant characteristics.

The document US 2020/0397313 (henceforth US20) presents systems, methods, devices, and techniques for estimating an ejection-fraction characteristic of a mammal. An electrocardiogram (ECG) procedure is performed on a mammal, and a computer system obtains ECG data that describes results of the ECG over a time period. The system provides a predictive input that is based on the ECG data to an ejection-fraction predictive model, such as a neural network or other machine-learning model. In response, the ejection-fraction predictive model processes the input to generate an estimated ejection-fraction characteristic of the mammal. The system outputs the estimated ejection-fraction characteristic of the mammal for presentation to a user.

The document WO 2019/046,004 (henceforth WO19) provides an Integrated Cardiorespiratory (ICR) System for continuous Stroke Volume (SV) measurement using a wearable device comprising a plurality of acoustic sensors. The ICR system performs signal processing computations to characterize cardiac acoustic signals that are generated by cardiac hemodynamic flow, cardiac valve, and tissue motion, and may use advanced machine learning methods to provide accurate computation of SV.

The document WO 2016/207,863 (henceforth WO16) presents a method and system to facilitate monitoring and/or evaluation of disease or physiological state using mathematical analysis and machine learning analysis of a biopotential signal collected from a single electrode. The exemplified method and system create, from data of a singularly measured biopotential signal, via a mathematical operation (i.e., via numeric fractional derivative calculation of the signal in the frequency domain), one or more mathematically derived biopotential signals (e.g., virtual biopotential signals) that is used in combination with the measured biopotential signals to generate a multi-dimensional phase-space representation of the body (e.g., the heart). By mathematically modulating (e.g., by expanding or contracting) portions of a given biopotential signal, in the frequency domain, the numeric-based operation gives emphasis or de-emphasis to certain measured frequencies of the biopotential signals, which, when coupled with machine learning, facilitates improved diagnostics of certain pathologies.

The Applicant conceived, tested, and incorporated the present invention in order to overcome the deficiencies of the state of the art and obtain the purposes and advantages mentioned above and explained below.

### SUMMARY OF THE INVENTION

The present invention is characterized in the independent claims, while the dependent claims describe other features of the invention or embodiments relating to the main inventive idea.

This invention presents a waterproof portable device for gathering and monitoring of ECG data of an individual, wherein the device has a Y configuration defined by a first arm extension, a second arm extension, a third arm extension and a housing. Each one of the first, second and third arm extensions are rigidly connected to the housing through one end or extremity and contain an electrode connector on the other end or extremity. The first and the second arm extension are positioned apart from each other by an angular distance β, the second and the third arm extension are positioned apart from each other by an angular distance α, the first and the third arm extension are positioned apart from each other by an angular distance γ, with α+β+γ=2π. The housing contains a button on its top portion and an electrode connector on its bottom portion, and holds an ECG board, a Li-Ion battery, with a maximum capacity of 7-day battery charge of uninterrupted use, without needing a recharge. The housing also contains a wireless communication system, a BLE antenna, a shielding against EMI, and an internal HD, with a storage capacity of 2 Gb. The combination of high storage capacity and battery charge makes the portable device of the presented invention suitable for a Holter and/or Event Monitoring exam.

In one embodiment, the housing contains additionally a wireless charging coil, and the shielding against EMI comprises a ferrite sheet or a ferrite plate.

The portable device is configured to work on both offline and online mode, with an automatic transition from online to offline mode. The online mode starts by pairing the portable device through the BLE antenna to a second device that contains a communication software, such that the portable device is configured to upload the stored data into the backend software and free up the internal HD memory. Alternatively, the online mode starts by placing the portable device over a wireless charger, such that the portable device is configured to search and pair with the second device that contains the communication software and it is configured to upload the stored ECG data in the backend software and free up the internal HD memory. The offline mode starts automatically by unpairing the portable device through the BLE antenna to the second device that contains the communication software, such that the portable device is configured to store the ECG data in the internal HD. Alternatively, the offline mode is defined when setting the exam by the person requesting the exam.

In one embodiment, the communication software is a native mobile app. In another embodiment, the communication software is a hybrid mobile app. In another embodiment, the communication software is an embedded app in a proprietary device (docking charger or station). The backend software is, in one embodiment, a web app.

The button on the housing is configured to generate a data identifier when held down, such that it is pushed by an individual wearing the ECG portable device, and the ECG data collected at the same day and time as the data identifier receives a marker.

This invention also brings a process for analyzing, interpreting, and reporting ECG data. This process comprises at least the steps of (a) preparing an ECG exam through a first device containing a software; (b) connecting the first device to a portable ECG device; (c) setting up the portable ECG device on a patient's chest; (d) collecting the ECG data using the portable ECG device; (e) uploading the data from step “d” to an artificial intelligence backend software through a communication software; (f) processing and classifying the data using a machine learning algorithm; (g) analyzing the classification provided in step “f” and performing a second classification of data; and (h) providing a signed medical report for the ECG data collected.

The ECG data is collected by the portable device using 1, 2 or 3 channels, each Lead corresponding to a difference of the data between two channels. The data from each Lead is stored separately from the data of the others, allowing for an independent analysis of the Leads. Besides, in case of a data identifier, i.e., in case the individual presses the button, the correspondent data is stored along with the ECG data, so that the ECG data collected at the same day and time as the data identifier receives a marker.

In one embodiment, the process of the current invention uses the portable device operating in the offline mode. In this mode, the process is confined to steps (a) and (b) in a loop, happening continuously and sequentially until the storage capacity is reached or until the portable device enters the online mode.

In another embodiment, the process of the current invention uses the portable device operating in the online mode. In this mode, the data already contained within the internal HD and the new data is uploaded to the backend software. At every received data, the backend software verifies the signal quality of the ECG data and either requests for an adjustment of the portable device or requests for a replacement of electrodes for signal improvement. The backend software qualifies the received data signal as standard or substandard, wherein the substandard quality corresponds to a signal drop, and the backend software displays a notification in a software interface requesting the electrode replacement, specifying the channel with the poor signal or electrode to be replaced.

The backend software groups the uploaded data in a set of clusters, wherein a machine learning algorithm classifies the input data into Normal, “N”, Ventricular, “V”, Supraventricular, “S”, Unknown, “Q” and Artifact, “X”, each cluster being composed by a set of data of the same classification and the data of each Lead being analyzed individually and independently.

In one embodiment, a medical doctor evaluates the clusters, wherein the medical doctor removes from the clusters the input data wrongly classified, and the backend software updates its classification analysis through reinforcement learning.

In one embodiment, the data classified by the backend software is displayed in a software interface, wherein the data is presented at least in the form of statistical occurrence, countable events, color identification, ECG signal and comments within the ECG signal, and the data is calculated for each one of the 3 Leads individually and, additionally, as a resultant single Lead (name QuoreLead).

In another embodiment, the backend software displays the heartbeat information in a software interface, wherein the data is presented at least in the form of countable events and graphical distribution, and the graphical distribution contains the actual heartbeat and a maximum, minimum, and average of a measuring period.

The backend software generates a medical report that contains at least the resultant single Lead ECG signal (QuoreLead), the data marker over the ECG signal and the major cardiac events. In one embodiment, the report contains additional comments included by the medical doctor. The report is printable.

In one embodiment, the process of the current invention is specially adapted to be applied with the portable device of the current invention.

### OBJECTIVES OF THE INVENTION

One objective of this invention is to present a portable device that is ergonomic, light, comfortable and of ease usability for the individual or patient, improving the quality of the collected ECG data.

It is another objective of this invention to present a portable ECG device interactive, allowing the patient to easily report symptoms by simply pressing a button or recording an annotation in the communication software. This new concept optimizes the analysis by the medical doctor with the inclusion of these moments of interest as data markers on the ECG data.

It is another objective of the present invention to present a process for ECG data analysis that is optimized, reducing considerably the chance of a possible misdiagnosis by analyzing both the resultant ECG signal (Quorelead) and the individual contribution of each Lead to the ECG signal.

It is another objective of the present invention to present an artificial intelligence algorithm with a reinforcement learning method to improve the ECG analysis, with an active contribution of the medical doctor to the training process and algorithm choice. More specifically, it presents a set of algorithms already existing in the system and allows the training by the medical doctor him(-) or herself.

### BRIEF DESCRIPTION OF THE FIGURES

These and other features of the present invention will become evident from the following description of some embodiments, given by way of non-restrictive example with reference to the accompanying drawings, in which

FIGS. 1A and 1B show a planar view of the portable device according to this invention,

FIG. 2 shows top and bottom perspective views of the portable device according to this invention,

FIG. 3A shows a perspective bottom view from the interior of the portable device of this invention

FIG. 3B shows a perspective top view from the interior of the portable device of this invention

FIG. 4A shows a workflow for one embodiment of a preparation and installation of the device for a Holter exam according to the process of this invention,

FIG. 4B shows a workflow for one embodiment of the exam process and data uploading according to one variation of the process of this invention,

FIGS. 5A to 5C show images of the layout of the communication software installed in the exam requestor device for the process of this invention,

FIGS. 5D to 5H show images of the layout of the communication software installed in the patient's device for the process of this invention,

FIGS. 6A to 6F show images of the layout of the backend software for the process of this invention,

FIG. 7 shows the wearing process of the portable device on the individual or patient's chest.

For ease of understanding, the same reference numbers have been used, whenever possible, to identify identical common elements in the drawings. It is understood that elements and characteristics of a modality can be conveniently incorporated into other modalities without further clarification.

### DETAILED DESCRIPTION OF THE INVENTION

We will now refer in detail to the various embodiments of the present invention, one or more examples of which are shown in the accompanying drawings. Each example is provided by way of illustration of the present invention and is not to be construed as a limitation of this invention. For example, characteristics presented or described insofar as they form part of one modality may be adopted in (or be in association with) other modalities to produce another modality. It is understood that the present invention is to include all such modifications and variants.

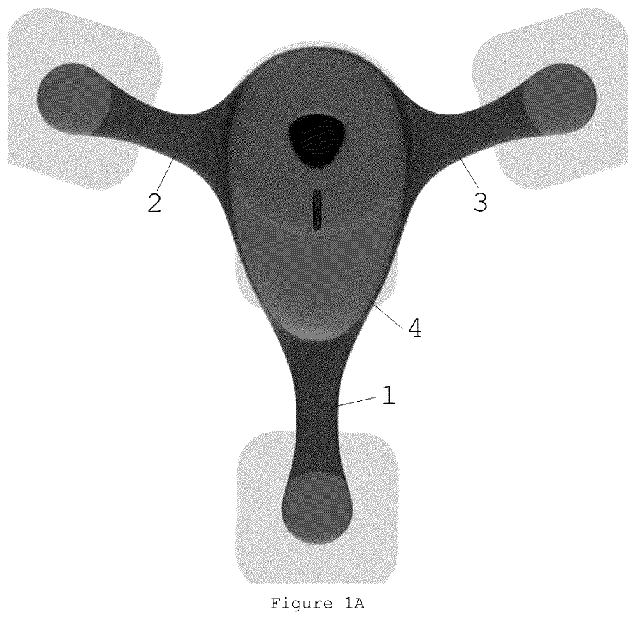

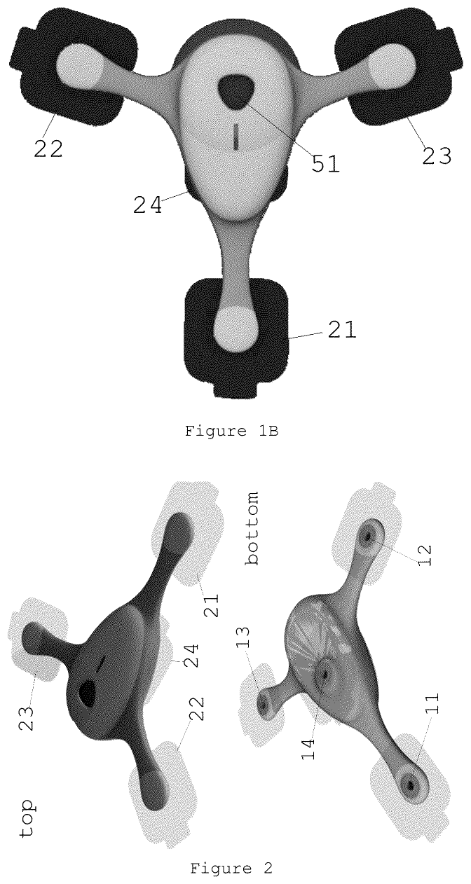

FIGS. 1A, 1B and 2 show a planar view of the portable device according to this invention. The portable ECG device has an Y configuration, being this shape optimized for a better reading of the heart electrical signal. The Y shape is defined by three arms (1, 2, 3) rigidly connected through one extremity to the housing (4), where this connection is made such that the angular distance (α, β, γ) between two consecutive arms provide an optimized reading of the ECG signal. The first and second arms (1, 2) are apart from each other by an angular distance β, the second and third arms (2, 3) are apart from each other by an angular distance γ, and the third and first arms (3, 1) are apart from each other by an angular distance α. Besides, all three arms (1, 2, 3) are contained within the same plane, i.e., they are coplanar, and α+β+γ=2π. The extremity of each arm (1, 2, 3) contains an electrode connector (11, 12, 13) to connect to an electrode (21, 22, 23). A fourth electrode connector (14) is found on the bottom part of the housing (4) and connects to a fourth electrode (24).

The portable ECG of this invention is based on a 3-Lead ECG, with a first electrode (21) corresponding to the reading from the left leg (LL), one electrode (22) corresponding to the reading of the right arm (RA) and one electrode (23) corresponding to the reading of the left arm (LA). Besides, the fourth electrode or reference electrode (24) is placed over the sternum line. The placement of the portable device over or near the sternal midline considerably improves the ability of the ECG device to sense cardiac electric signals, particularly the P-wave. Additionally, the compact size and ergonomic shape of the device results in a comfortable long period usage.

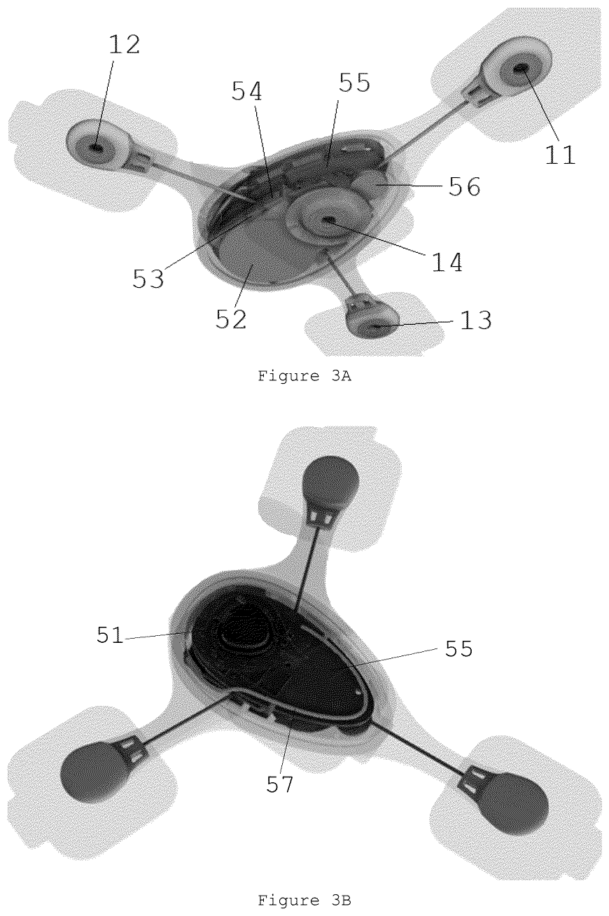

FIGS. 3A and 3B show in more detail some of the internal components of the portable device, placed in the housing (4). Among these components, the housing (4) holds in its interior an ECG board (55), a battery (54), a wireless communication system (56), a BLE (Bluetooth) antenna (57), an internal HD (58), a shielding against EMI (53), and a wireless charging coil (52).

The portable ECG device of this invention accepts wireless charging as well as wired charging. The wireless charging starts by placing the device on top of a docking charger or station. The battery (54) is of Li-Ion type with a maximum charge capacity for a 7-days uninterrupted use, being suitable for a Holter exam.

The portable device of this invention is also waterproof, allowing continuous ECG signal monitoring, for example, when showering. Besides, due to the fact that it is tightly closed, the internal components are protected from environmental damage, like rain and dust. The presence of a wireless charging coil (52) does not affect the ECG performance and neither interfere with nor suffer interference from external magnetic fields. The shielding against EMI (53) protects the device from emitting an electromagnetic field and protects the device itself against electromagnetic fields, and it is a ferrite shielding. This shielding (53) can be used in a plate form (ferrite plate), sheet form (ferrite sheet) or even as a coating.

The internal HD (58) has a maximum storage capacity of 2 Gb, and it is used as a storage for the offline mode and a backup for the online mode. More specifically, the ECG data collected is stored in the internal HD (58) on both modes, and the data remains locally stored until it is successfully uploaded to the system. This upload happens automatically by connecting the device to a backend software through the BLE antenna (57) or the wireless antenna (56), and this connection can be initiated by the individual wearing the device or be triggered by placing the device on the charging station. Once successfully uploaded, the data is removed from the internal HD, freeing space for another set of data.

The housing (4) also includes, placed on its top part, a button (51) connected to the ECG board (55) for data entry by the individual or patient. This data entry corresponds to a “pressing” of the button by the patient, and it marks the data the patient wishes the medical doctor to look at with further attention. For example, the patient could feel a palpitation, blood pressure dropping, dizziness or any other physical symptoms or discomfort that he/she believes is of importance.

The data is stored without treatment or processing in the device, being processed only by the backend software, once uploaded. The backend software according to this invention can be a native mobile app, a hybrid mobile app or a web app.

The process of analyzing the ECG data can be divided into at least 8 steps, being (a) preparing an ECG exam through a first device containing a software; (b) connecting the first device to a portable ECG device; (c) setting up the portable ECG device on a patient's chest; (d) collecting the ECG data using the portable ECG device; (e) uploading the data from step “d” to an artificial intelligence backend software through a communication software; (f) processing and classifying the data using a machine learning algorithm; (g) analyzing the classification provided in step “f” and performing a second classification of data; and (h) providing a signed medical report for the ECG data collected.

It is important to highlight that the process involves three principal elements: an ECG portable device, a communication software, and a backend software. The ECG device is responsible for collecting the ECG data of the patient and either store it internally in an internal HD or upload it to a software or a cloud-based storage unit. The communication software is responsible for the communication between the ECG device and the backend software, and it is presented in the form of an app. In different embodiments, the communication software can be a native mobile app or a hybrid app, when installed in a mobile device, or an embedded app when installed in a proprietary device, such as a docking charger or station. The exam is prepared through the communication software installed in a first device (although there is also an operation mode that allows the patient to self-start the exam), for example, a smartphone, belonging to the medical doctor or operator. A second version of the communication software, or a different interface, is installed in a second device, for example, a smartphone, belonging to the patient. The backend software is accessible only by the medical doctor or operator, comprising a machine learning algorithm for analysis and report of the ECG data.

In another embodiment, the exam is prepared through the communication software installed in a second device, for example, a smartphone, belonging to the patient. The communication software allows the self-installation of the ECG portable device with the initiation of the exam by the patient him(-) herself. For example, the medical doctor/operator can remotely prepare an exam and configure it to be taken through the ECG device with the patient. An invite is sent by the doctor/operator, through the communication software, from the first device to the second device and, from this point on, the ECG device is initiated, paired with the second device, and installed on the patient's chest by the self. In another embodiment, the ECG device is handed over by the medical doctor/operator to the patient to start the self-installation and perform the exam. Once the exam is complete, the medical doctor gains access to the ECG data, performs the analysis and generates the medical report.

In one embodiment, the communication software installed in the first device has an interface specially developed to prepare the exam and the communication software installed in the second device has an interface specially developed to easily guide the patient through the exam. In another embodiment, the communication software contained in either the first or the second device has the same interface. In another embodiment, the communication software installed in either the first or the second device changes interface automatically through the identification of the user-medical doctor/operator or patient.

The first step, (a), comprises the preparation of the exam to be performed in the patient. This preparation is made by the medical doctor through the communication software installed in the first device. The exam is attached to a specific patient though an identification number. In one embodiment, the identification is a unique identifier of the patient, for example, SSN (social Security Number) in the USA and CPF (Cadastro de Pessoa Física) in Brazil. In another embodiment, the identification number is a medical registry belonging to said person. In another embodiment, the identification number is a personal unique numeric combination, generated on the first time using the device and process described in this invention. Alternatively, the identification number can be a random non-personal identifier. The choice for the identification number is associated with the chosen option for the exam. If the exam is going to be performed locally, in the presence of the medical doctor, the exam can be performed regardless of a personal identification number. However, if the patient opts to perform a remote exam or a self-installation exam, a personal identification number might be required.

The communication software also requests a couple patient's information significant for the exam and the medical report, for example, the presence of pacemaker or ICD and smoking habits. It also requests the symptoms that led to the exam, such as dizziness, fainting and palpitations, and the major reason for the doctor to be requesting that exam. For example, the medical doctor might be interested in confirming an arrythmia, identifying an ischemia, documenting an anti-arrhythmic or anti-ischemic event, or even predicting a cardiac event by collecting data in the patient's cardiac history.

The exam can run, without recharging the device, for a period from 5 minutes up to 7 days. More specifically, the medical doctor can choose the duration of the exam in minutes—for example, 5, 10, 15, 30, 45 or 60—, in hours—for example, any duration between 2 and 24 hours, or in days—for example, any duration between 2 and 7 days.

The communication software automatically sets up if the exam will consider the readings from one, two or all three channels. This choice depends on the purpose and/or duration of the exam prepared by the medical doctor. The channels are defined as C1, C2 and C3, where C1 reflects the reading between the electrodes of the right arm, “RA”, and the left arm, “LA”, named Lead1, C2 reflects the reading between the electrodes of the right arm, “RA”, and the left leg, “LL”, named Lead2, and C3 reflects the reading between the electrodes of the left arm, “LA”, and the left leg, “LL”, named Lead3. For example, the medical doctor can request the reading from channel C2 only—“RA-LL”—or request the readings from two channels, C1 and C2—“RA-LA” and “RA-LL”—, or even request the readings from all three channels, collecting the data from all three Leads.

Finally, the medical doctor can choose if the exam will be performed online or on offline mode, with the ECG device being installed on the patient's chest by the doctor or by the patient him(-) or herself. An online exam requires a second portable device belonging to the patient, such as a smartphone, containing the communication software installed in it.

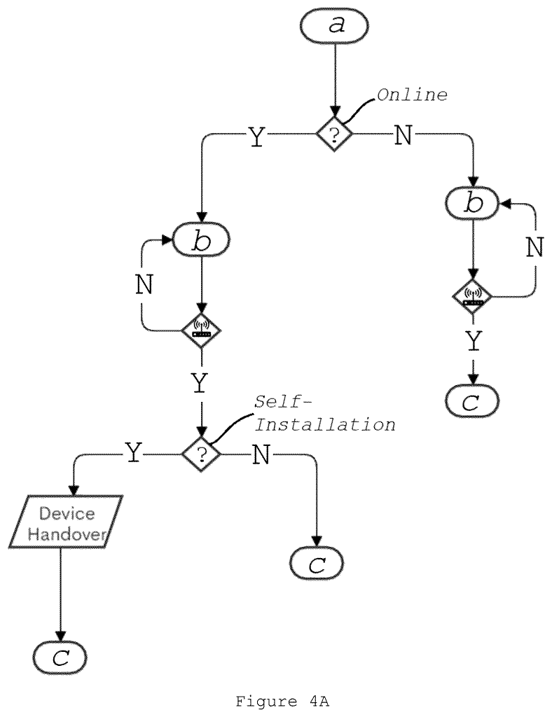

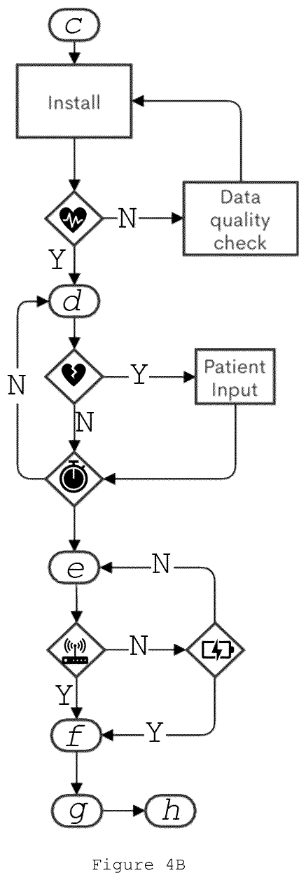

Once the exam is created by the medical doctor, it is attributed to a specific ECG device by pairing the first device to the ECG portable device through the communication software, step (b). As can be seen in FIG. 4A, the connection attempt is repeated until it is successful. This first connection happens between the first device and the ECG portable device with the purpose to properly set one specific device to perform that exam at that moment. This step (b) also represents the connection between the ECG portable device and the second device, when it is applicable. So, for one embodiment, it is possible that the process presents a sequence of (b) steps, for a local online exam, or a second (b) step placed between steps (c) and (d), for the online remote exam. The flowchart displayed in FIG. 4A should not be considered as restrictive of the invention, but as one example for one embodiment of this invention.

Through the communication software, the portable ECG device receives the input to start the exam and it begins to send the real-time data to the backend software. Should a loss of connection occur during the exam once the exam is complete, the communication software connects with the backend software to upload the remaining ECG data. Alternatively, remaining ECG data may be sent to the backend software using a docking charger or station that has the communication software installed. On the other hand, an offline exam does not require the second device. The exam starts through an input sent through the communication software installed in the first device, belonging to the medical doctor, and the ECG data is stored in the internal HD of the ECG device. Alternatively, if the exam is being taken remotely by self-installation, the exam starts through an input sent through the communication software installed in the second device, belonging to the patient. The ECG data is uploaded once the ECG device is placed over a proprietary device, such as a docking charger or station, or in the event of a posterior connection between the ECG device and the communication software through either the first or second devices. The online exam behaves the same way if performed either locally, with the ECG device being placed by the medical doctor, or remotely, through self-installation.

The versatility of the ECG device of this invention allows it to present itself as a POCT (Point Of Care Testing), once it is an automatized, easily operable and portable device that confers the patient the ability to perform complex laboratorial exams within the comfort of their home. Alternatively, it is easily applicable in the ambulatorial environment, in hospitals and urgent healthcare units, and in the medical doctor's office.

### Description of One Embodiment for an Online ECG Exam

One embodiment of the process from this invention is further described in here, and it considers that the exam is performed online through a self-installation. This description is an exemplification of one embodiment of this invention and should not be considered as restrictive.

FIG. 4B brings an exemplification of the embodiment further described in detail. The patient can start the exam while in the presence of the medical doctor, on his/her way home or in a place where he/she feels comfortable to perform the exam. If initiated while with the medical doctor, a self-installation is not required, and the exam can start by simply pairing the communication software from the second device with the portable ECG device. On the other hand, if the patient opts for initiating the exam later that day or once he/she arrives home, the medical doctor sends an invite to the patient through the communication software. Said invite can be sent as an e-mail, a SMS, a QR Code or by sharing an invitation link through an app of the patient's choice.

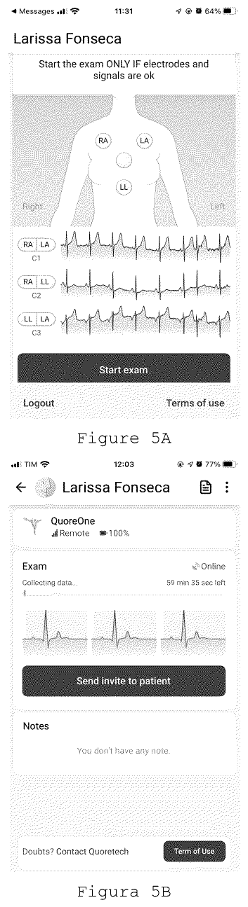

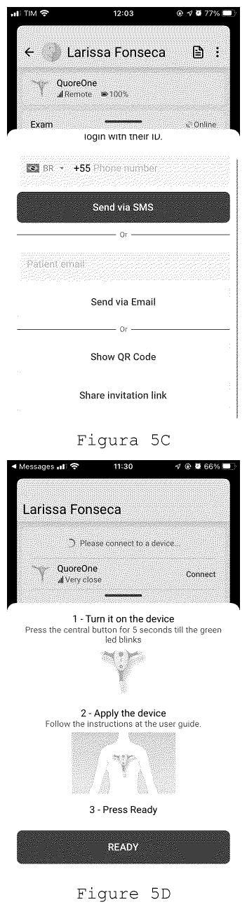

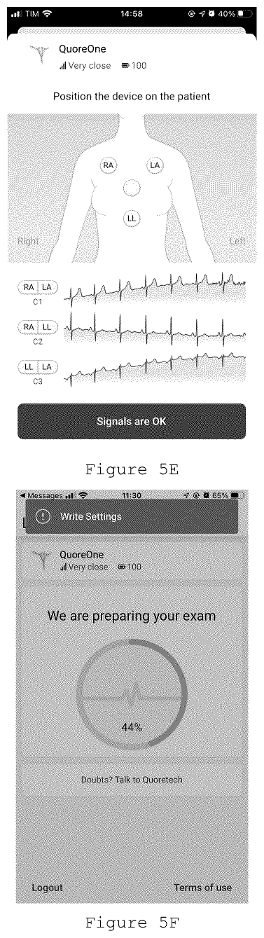

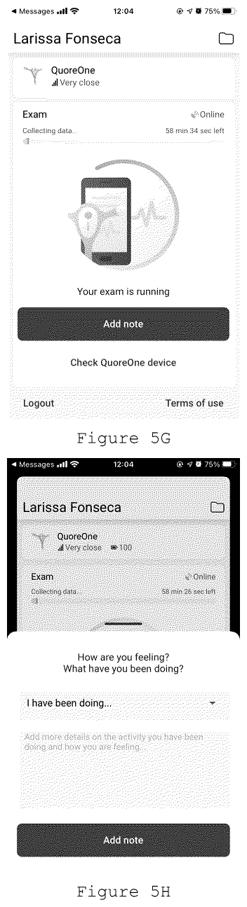

The patient turns on the ECG device, installs it on his/her chest accordingly, step (c), and pairs the ECG portable device to the communication software through the second device (FIG. 5D). Prior to start, the communication software verifies the quality of the signals collected by the electrodes and, if there is a bad reading, a replacement of the electrode is indicated. FIG. 5E shows an embodiment where the signal quality is indicated through the channels or Leads. In this embodiment, a malfunctioning electrode implicates two channels with poor quality readings. In another embodiment, the communication software indicates the specific electrode that needs to be replaced or adjusted.

If the received data has an identified signal dropping, the backend software sends a notification to the frontend, displaying a message requesting the replacement of an electrode (21, 22, 23, 24). According to one embodiment, the message contains an indication of the electrode that needs replacement. For example, if the signal from the reference electrode (24) is bad or substandard, the message informs that the fourth electrode (24) needs to be replaced by a new one. On the offline mode, a poor signal is signaled by the ECG device through the LED present near the button. If the ECG device identifies a poor signal, the LED changes color. Under regular functioning conditions, the LED light will present a green color and a deviation from a regular functioning condition is presented by a blue color LED. For example, if the ECG device identify identifies a poor signal, the LED starts blinking in a blue color. In another example, while placed over a charger or docking station, it is possible to identify if the ECG device is properly charging through a blinking blue color LED.

Once the signals are good on all three channels, the patient starts the exam by pressing the virtual button on the communication software, giving an input to the ECG portable device (FIG. 5E, 5F). The ECG device collects the ECG data, step (d), and keeps it in its internal HD until the end of the exam.

During the exam, the patient can provide information regarding any symptom or activity being performed. The ECG device has a button on its upper surface that, when pressed, gives an input data stored within the collected ECG data. If the patient feels an abnormal heartbeat, some dizziness, faints or identifies any other symptom that he/she believes to be relevant for the exam, he/she can add this information by simply pressing the button. This action creates an event that will be later included in the ECG data presented to the medical doctor. Additionally, the patient can include an annotation or a detailed explanation of the identified symptom or the activity performed during the exam. This annotation is made through the communication software (FIG. 5H) and can be included at any point during the exam or even once the exam is done, prior to the analysis and report performed by the medical doctor.

Once the exam is done, the collected data is uploaded to the backend software through the communication software, step (e). If the ECG device is still paired with the second device, the upload is performed automatically. Otherwise, if the second device loses connection somehow, the upload will start once the connection is restored or once the ECG device is placed over a docking charger or station containing the communication software.

### Description of the Backend Software Functioning

The ECG data and patient inputs and notes uploaded during step (e) is analyzed and processed by the backend software that, in one embodiment, is a web app. The backend software is accessible only to the medical doctor, who, with the support of the machine learning algorithm with a reinforcement learning base, analyzes and documents the patient's ECG results.

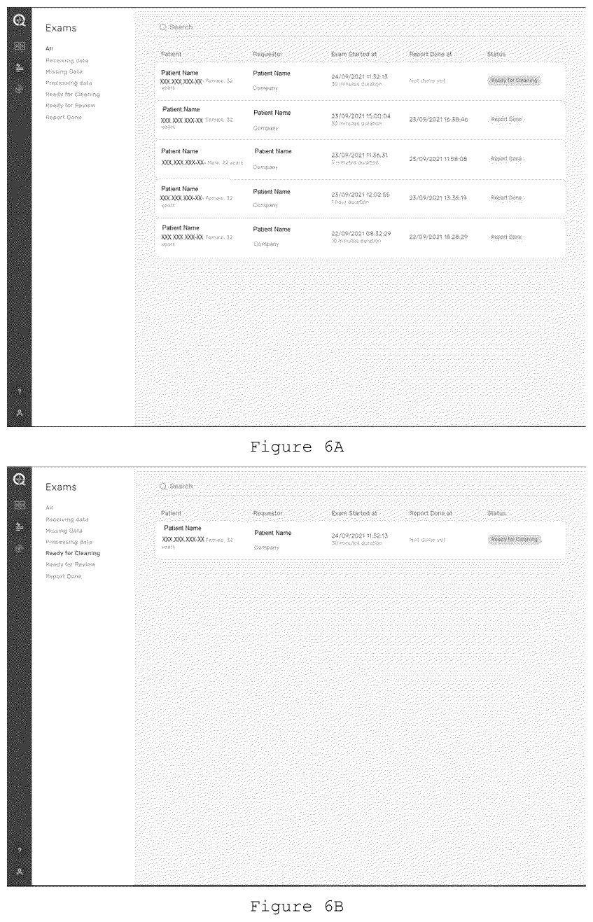

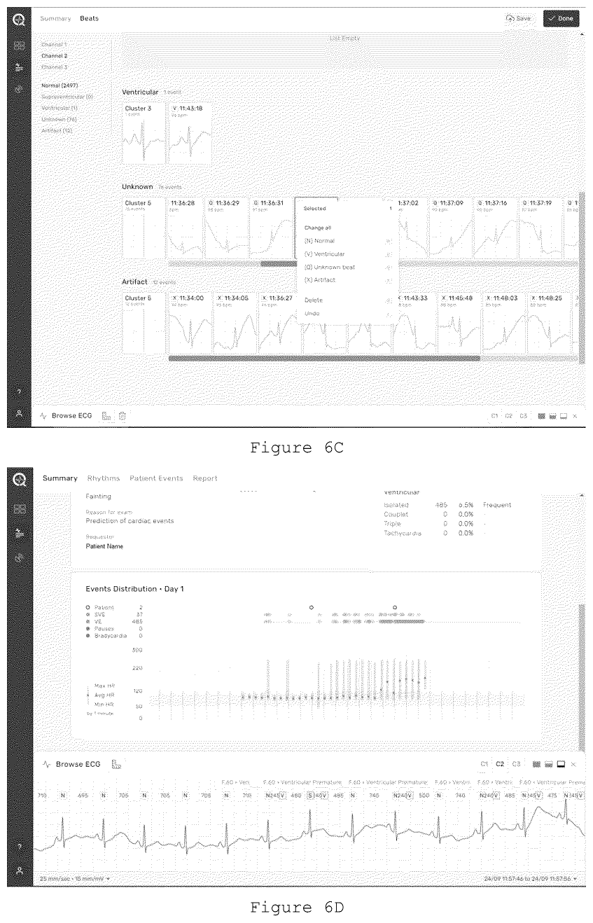

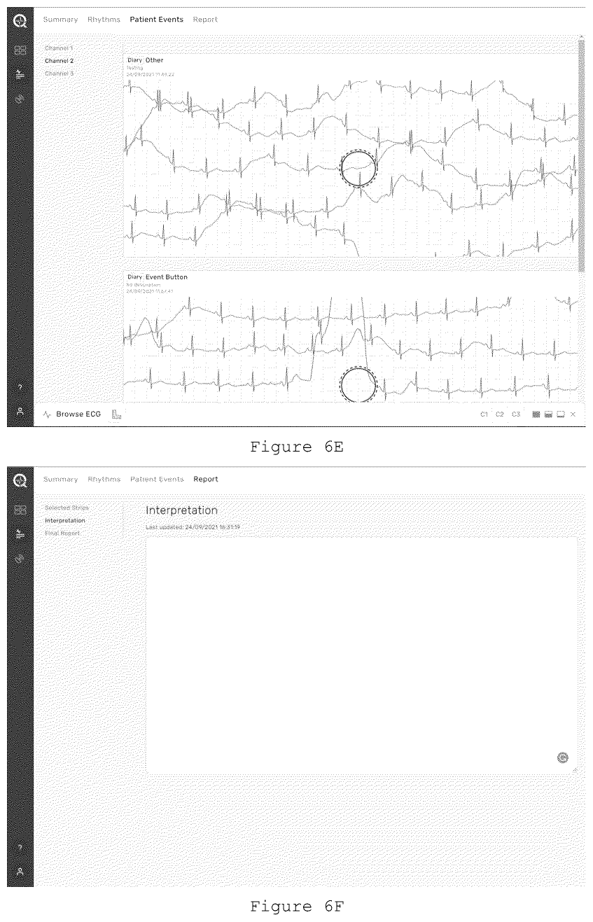

The backend software has a Graphical User Interface (GUI) to manage interaction with the system. The GUI is customizable to better fit the user's preferences allowing a more comfortable experience. For example, the user or, in this case, the medical doctor, can choose between a light and a dark theme and the preferred language for display, where English is the standard language. Regarding the ECG information, the GUI presents in its layout the set of data on each stage of the exam processing (FIG. 6A). It also contains a Live Monitor to display the medical exams being performed in online mode.

The data received by the backend software, step (f), is pre-processed using a machine learning algorithm with a reinforcement learning/deep learning base method. The algorithm evaluates the data and separates into clusters or sets of data. The backend software offers at least three options of algorithms to perform the analysis: the first algorithm, named “QT Golden”, contains cleaning techniques performed by medical doctors specialized in heart diseases that are highly recognized and acclaimed in their field. The second algorithm available, named “Personal”, is an algorithm offered to the medical doctor performing the exam and can be personalized. For example, it can be trained for a set of ECG signals of people reporting a “VE” or ventricular event, such as a ventricular fibrillation. Thus, the algorithm is specially adapted to better identify and qualify ventricular ECG signals. The third algorithm, named “General”, is composed of a combination of the two previous ones, encompassing the training performed by each medical doctor using the software.

The data identified as heartbeat, also known as QRS Complex, is classified according to its waveform into Normal, “N”, Ventricular, “V”, and Supraventricular, “S”, and the classification is performed using as basis the information provided by the American Heart Association (AHA). The remaining ECG data that is identified as a heartbeat, but it is not initially categorized into either “N”, “V” or “S”, is classified as Unknown, “Q”, defining a fourth cluster. One last cluster is generated, named as Artifact, “X”, containing the remaining data that is not a heartbeat waveform. This “X” cluster contains, for example, noise, electromagnetic interference (EMI) or even bad quality signals. As one example, data collected using a damaged electrode might result in a squared-like waveform ECG signal, being stored into the “X” cluster.

This first classification is provided to the medical doctor for further analysis or “Cleaning” of the clusters (FIG. 6B). The cleaning process, corresponding to the step (g) The data within each cluster is accessible to the medical doctor or professional, who can interact with the software by updating the data. For example, the medical doctor can inform that a signal classified as “V” is, in fact, “N” result, changing its classification and, therefore, updating the clusters (FIG. 6C). This action also updates the algorithm information, training the algorithm according to a reinforcement learning method.

According to one embodiment, the process in this invention allows the developing of optimized algorithms that can be trained by a specific group of medical doctors or a single medical doctor

Once the clusters are cleaned by the medical doctor, the data is ready to be reviewed. In this step, corresponding to the final step (h), the algorithm performs a second evaluation of the data. In this new analysis, the algorithm creates a resultant of the leads analyzed (QuoreLead), producing a final classification to that QRS complex. Next, it also identifies the different rhythms observed in the ECG data contained in clusters “V”, ventricular, “S”, supraventricular, and “N”, normal. For example, the algorithm can identify a supraventricular event, “SVE”, or a ventricular event, “VE”. As examples, it can identify a Tachycardia (speeding up of the heartbeat rhythm) or a Bradycardia (slowing down of the heartbeat rhythm), pauses (a period higher than a predefined time variable with no heartbeat associated), a ventricular premature complex, a supraventricular complex, an ectopic atrial rhythm, a fibrillation (atrial or ventricular), an atrial flutter, an accelerated idioventricular rhythm, a short or prolonged PR interval, an AV block of Mobitz type I and II, of varying conduction, of high-grade or of third-grade, or even an AV dissociation.

It is also in step (h) that the backend software indicates the events added by the patient during the exam. The notes are included as attachments over the ECG waves graphic, making it easily identifiable. Besides, it allows a quicker analysis by the medical doctor of the cardiac event once the information provided by the patient is easily accessible and already attached to the ECG data. For example, if the patient identifies a symptom and presses the button, it will be printed on the ECG data and the medical doctor can assertively evaluate if there is an anomaly or not (FIG. 6D).

FIG. 6E shows the identification of a patient event on the ECG data, providing a closer look to the ECG signal at the moment highlighted by the patient. As it can be seen in FIG. 6E, there are at least two types of event inputs or entries on the patient's “Diary”. The first input type is provided by the button on the ECG device that, when pressed, creates an event with the date and time. The second input type is a note written by the patient through the communication software, informing any symptom identified by the patient or activity performed during the exam.

In one embodiment, the data is presented by the backend software in a software frontend layout, GUI, in the form of statistical occurrence, countable events, color identification, ECG signal and comments within the ECG signal, where the data is presented for each one of the 3 Leads individually and, additionally, as a resultant single Lead (QuoreLead). The heartbeat information is also displayed in the layout, where the data is presented at least in the form of countable events and graphical distribution, that contains the actual heartbeat and a maximum, minimum, and average of a measuring period.

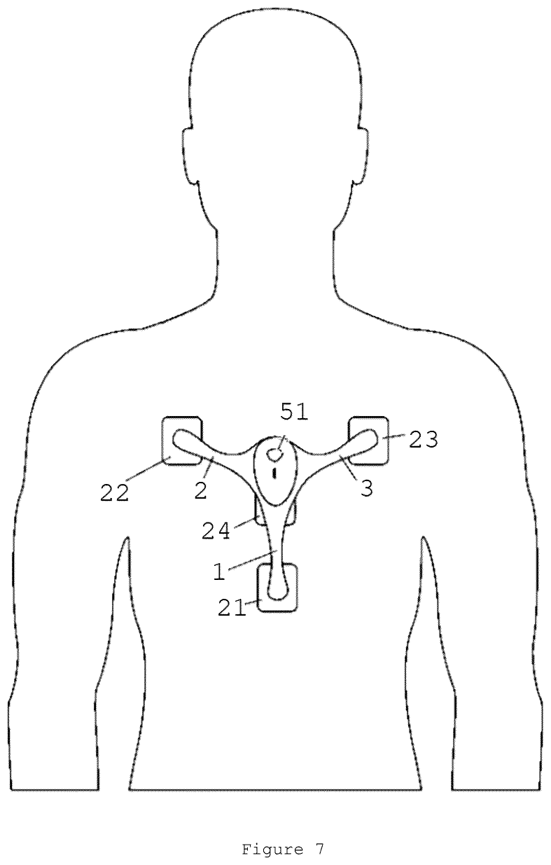

The analysis is also condensed into a printable report that contains at least the resultant single Lead ECG signal (QuoreLead), the data marker over the ECG signal and the major cardiac events. Additionally, the medical doctor can include text input into the ECG signal to appear in the printable report. The final report includes, also, an entry for the Interpretation of the results by the medical doctor, as shown in FIG. 7. At last, the backend software presents a final report containing the information marked by the doctor, the entries of the patient and any other highlighting considered important by the medical doctor. It also includes the ECG signal and any identified cardiac event. The final report can be signed digitally by the medical doctor or manually once printed. Once finalized and signed, the final report is sent to the communication software contained in the second device, belonging to the patient.

1. A portable device for gathering and monitoring of ECG data of an individual, wherein the device has a Y configuration defined by a first arm extension (B1), a second arm extension (B2), a third arm extension (B3) and a housing (A), wherein
the second and the third arm extension are positioned apart from each other by an angular distance α,
the first and the second arm extension are positioned apart from each other by an angular distance β, and
the first and the third arm extension are positioned apart from each other by an angular distance γ, with α+β+γ=2π,
each one of the first, the second and the third arm extension are connected to the housing through a first extremity, and contains an electrode connector on a second extremity, and
the housing contains a button on its top portion and an electrode connector on its bottom portion, and holds an ECG board, a battery, a wireless communication system, a BLE antenna, an internal HD, and a shielding against EMI.

2. The portable device of claim 1, wherein the housing contains additionally a wireless charging coil.

3. The portable device according to claim 1, wherein the shielding against EMI comprises a ferrite sheet or a ferrite plate.

4. The portable device according to claim 1, wherein each one of the first, the second, and the third arm extension are rigidly connected to the housing through the first extremity.

5. The portable device according to claim 1, wherein each one of the first, the second and the third arm extension are movably connected to the housing through the first extremity.

6. The portable device of claim 1, wherein the internal HD has a storage capacity of 2 Gb.

7. The portable device according to claim 1, wherein in that the portable device is configured to work on online mode and on offline mode.

8. The portable device of claim 7, wherein, in the online mode, the portable device is configured to connect through the BLE antenna to a second device containing a software, such that
the portable device is configured to upload the data into the software,
the software is a native mobile app, a hybrid mobile app or a web app.

9. The portable device of claim 7, wherein the portable device is configured to starts automatically the offline mode by unpairing the portable device through the BLE antenna to the second device that contains the software, such that
the portable device is configured to store the ECG data in the internal HD.

10. The portable device according to claim 1, wherein in that it is waterproof.

11. The portable device of claim 1, wherein the battery is a Li-Ion battery.

12. The portable device of claim 11, wherein the battery has a maximum capacity of a 7-day battery charge of uninterrupted use.

13. The portable device according to claim 1, wherein it is configured to perform a Holter exam.

14. The portable device according to claim 1, wherein the button is configured to generate a data identifier when pushed, such that
the button is pushed by an individual wearing the ECG portable device, and
the ECG data collected at the same day and time as the data identifier receives a marker.

15. A process for analyzing, interpreting, and reporting ECG data wherein the process comprises:
a. preparing an ECG exam through a first device containing a software,
b. connecting the first device to a portable ECG device,
c. setting up the portable ECG device on a patient's chest,
d. collecting the ECG data using the portable ECG device,
e. uploading the data from step “d” to an artificial intelligence backend software through a communication software,
f. processing and classifying the data using a machine learning algorithm,
g. analyzing the classification provided in “f” and performing a second classification of data, and
h. providing a signed medical report for the ECG data collected.

16. The process of claim 15, wherein the backend software is a native mobile app, a hybrid mobile app, or a web app.

17. The process of claim 15, wherein the communication software contained within the first and the second device is a native mobile app or an embedded app.

18. The process of claim 15, wherein in “a”, a medical doctor
chooses between online local exam, online remote exam, and offline exam, wherein
the installation of the portable device can be performed by the doctor, for the online local exam or for the offline exam, or by the patient, for the online remote exam,
chooses the exam duration, with a minimum of 5 minutes and a maximum of 7 days, and defines the number of channels between one, two and three.

19. The process according to claim 15, wherein “b” comprises connecting the portable ECG device to the first device through a BLE or Bluetooth antenna.

20. The process according to claim 15, wherein in the online remote exam the medical doctor sends an invite to the patient to the second device, wherein said invite is a SMS or an e-mail.

21. The process according to claim 15, wherein in the online local exam the medical doctor sends an invite to the patient to the second device, wherein said invite is a QR Code.

22. The process according to claim 15, wherein “c” comprises the installation and signal verification of the portable ECG device, wherein
the portable ECG device is installed by means of 4 electrodes placed on the patient's chest, being one LL, one LA, one RA, and one grounded, and
the electrodes' signal is indicated in the app.

23. The process of claim 22, wherein the portable ECG device has a button, wherein
the button is configured to generate a data identifier when pushed, and
the button is pushed by the patient wearing the ECG portable device.

24. The process according to claim 15, wherein “d” comprises collecting the ECG data, wherein
the data of each Lead is collected and stored separately and independently from each other, and
a data identifier is stored along with the ECG data.

25. The process according to claim 24, wherein the data identifier can also be in form of a note, wherein the patient inputs said note through the second device.

26. The process according to claim 15, wherein in “e”, the collected and stored data is uploaded to the backend software, wherein
the data is copied from an internal HD to the backend software through the communication software,
the backend software verifies quality of the received data, and
the backend software sends an input to the portable ECG device allowing the internal HD to free up space.

27. The process according to claim 15, wherein in “e”, the collected and stored data is uploaded to the backend software, wherein
the data is copied from an internal HD to the backend software through the communication software,
the backend software verifies quality of the received data, and
the backend software sends an input to the portable ECG device requesting a new try to upload the data.

28. The process according to claim 15, wherein in “e”, the backend software qualifies the received data signal as standard or substandard, wherein
substandard quality corresponds to a signal drop, and
the backend software displays a notification in a software interface requesting an adjustment of the portable device or the electrode replacement.

29. The process according to claim 15, wherein in “e”, the collected and stored data is uploaded to the backend software by placing the portable ECG device on a docking charger or station.

30. The process of claim 28, wherein the backend software identifies a channel collecting the substandard data signal.

31. The process according to claim 15, wherein in “f”, the backend software groups the uploaded data in a set of clusters, wherein
a machine learning algorithm classifies input data into Normal, “N”, Ventricular, “V”, Supraventricular, “S”, Unknown, “Q”, and Artifact, “X”,
each cluster is composed by a set of data of the same classification, and
the data of each Lead is analyzed individually.

32. The process of claim 31, wherein the Unknown cluster, “Q”, contains ECG waves not previously identifiable by the algorithm, and the Artifact cluster, “X”, contains electrical waves that differ from ECG data, including noise, EMI and bad quality signals.

33. The process according to claim 15, wherein in “g”, a medical doctor evaluates clusters, wherein
the medical doctor removes from the clusters the input data wrongly classified, updating its classification, and
the backend software updates its classification analysis through reinforcement learning.

34. The process according to claim 15, wherein in “h”, the backend software displays classified data in a software interface, wherein
the data is presented at least in form of statistical occurrence, countable events, color identification, ECG signal, and comments within the ECG signal, and
the data is presented for each one of 3 Leads individually and, additionally, as a resultant single Lead.

35. The process of claim 15, wherein the backend software displays heartbeat information in a software interface, wherein
the data is presented at least in form of countable events and graphical distribution, and
the graphical distribution contains actual heartbeat and a maximum, minimum, and average of a measuring period.

36. The process of claim 15 wherein the backend software generates a medical report that contains at least resultant single Lead ECG signal, a data marker over the ECG signal and major cardiac events.

37. The process of claim 36, wherein the report contains additionally comments included by the medical doctor.

38. The process according to claim 15, wherein the report is printable.

39. The process according to claim 15, wherein the process is configured to use the portable device, wherein the device has a Y configuration defined by a first arm extension (B1), a second arm extension (B2), a third arm extension (B3) and a housing (A), wherein
the second and the third arm extension are positioned apart from each other by an angular distance α,
the first and the second arm extension are positioned apart from each other by an angular distance β, and
the first and the third arm extension are positioned apart from each other by an angular distance γ, with α+β+γ=2π,
each one of the first, the second and the third arm extension are connected to the housing through a first extremity, and contains an electrode connector on a second extremity, and
the housing contains a button on its top portion and an electrode connector on its bottom portion, and holds an ECG board, a battery, a wireless communication system, a BLE antenna, an internal HD, and a shielding against EMI.
